(19) United States
(12) Patent Application Publication
(10) Pub. No.: US 2005/0249355 A1
(43) Pub. Date: Nov. 10, 2005

(54) [FEEDBACK ACTIVE NOISE CONTROLLING CIRCUIT AND HEADPHONE]

(76) Inventors: Te-Lun Chen, Hsinchu City (TW); Jenn-Luen Chu, Taoyuan City (TW); Patrick Lee, Tauyuan City (TW); Hsueh-Chin Lin, Taipei City (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(21) Appl. No.: 10/604,511

(22) Filed: Jul. 28, 2003

(30) Foreign Application Priority Data
Sep. 2, 2002 (TW) 91213715
May 6, 2003 (TW) 92112279

(51) Int. Cl. A61F 11/06; G10K 11/16; H03B 29/00

(52) U.S. Cl. 381/71.14; 381/71.8; 381/74

(57) ABSTRACT

A feedback active noise cancellation headphone is disclosed. A plurality of microphone sensors are disposed in front of the left and the right speakers of the headphone for improving the sound reception quality of the microphone sensors, so that the active noise controlling circuit generates an inverse phase soundwave accurately for countering the low frequency noise, and further to improve the noise reduction performance of the active noise cancellation headphone.

### CROSS REFERENCE TO RELATED APPLICATIONS

This application claims the priority benefit of Taiwan applications serial no. 91213715, filed Sep. 02, 2002 and serial no. 92112279, filed May 06, 2003.

### BACKGROUND OF INVENTION

1. Field of the Invention

The present invention generally relates to a headphone, and more particularly to a headphone with microphone comprising a feedback active noise controlling circuit.

2. Description of the Related Art

Currently, the electronic products are getting popular, and the stereo product has become a significantly popular device for entertainment, wherein the headphone provides a convenient function for listening to the music. In order to provide a better stereo effect, a noise-cancellation method has to be applied to eliminate the environment noise accompanying together with the music sound. Based on the different noise-cancellation methods, the headphone is roughly classified into two different types, one is a passive noise-cancellation headphone and the other one is an active noise-cancellation headphone.

Since the passive noise-cancellation headphone relies solely on the sound isolating material to reduce the environment noise, its noise-cancellation capability mainly depends on the physical properties such as thickness, structure design and joint capability of the material used, thus the headphone is generally to be of large size and heavy weight. The material for making the passive noise-cancellation headphone almost do not have any capability to isolate the low frequency noise, for example, generated by the engine and the fan. In contrast, the active noise-cancellation headphone does not have this limitation, thus it is widely accepted by the consumers.

In the active noise cancellation headphones that are presently available on the market, it is common that one microphone sensor is disposed in front of the left speaker and the right speaker, respectively. With this approach, no matter where microphone sensors are disposed, only one microphone sensor senses the noise signal in front of the corresponding speaker, and therefore the performance of the microphone sensor is highly critical. Besides it is necessary to choose the microphone sensor that is highly sensitive and also expansive, in order to ensure the microphone sensor to maintain the original sound quality. It is also important to avoid the microphone sensor from easily getting damaged by the soldering process that would have adverse impact on the yield and cost when mass-produced. As the microphone sensors of the feedback active noise-cancellation headphone are disposed on the area in front of the speaker within a distance range of 0.5˜1 cm, there is a possibility of causing a serious near-field effect. Accordingly, even when a highly sensitivity microphone sensor is used, it is still adversely impacted by the near-field effect that occurs in front of the speaker, thus the noise reduction performance is significantly deteriorated.

Since the active noise controlling circuit in the conventional feedback active noise-cancellation headphone does not consider to separate the gain adjustment circuit of the audio input signal generated by the music apparatus such as the radio from the gain adjustment circuit of the noise perceiving signal obtained from the environment noise detected by the microphone sensors, and therefore the original spectrum of the music is impacted when the gain of the noise perceiving signal is adjusted for improving the anti-noise effect. Further, the low frequency cracked noise may be generated, or causes the problem of discomfort to the ear due to the sound level of the music bursts abruptly as the user turns on the power of the active noise controlling circuit while listening to the music.

### SUMMARY OF INVENTION

To solve the problems mentioned above and other defects, the present invention provides a feedback active noise cancellation headphone. With two or more than two microphone sensors disposed in front of the left and the right speakers of the headphone, the noise controlling circuit generates an inverse phase soundwave more accurately for countering the low frequency noise so that the active noise reduction performance of the active noise cancellation headphone can be effectively promoted. Thus, the sound reception quality of the microphone sensors can be effectively improved.

The present invention further provides a feedback active noise controlling circuit, wherein an adder whose gain can be separately adjusted, is used to respectively amplify an audio compensating signal from an audio compensating circuit and an environment noise signal from a bandpass controller, so as to reduce or eliminate the problem impacted by the adjustment of the anti-noise gain.

In order to achieve the above objectives and other advantages, the present invention provides a feedback active noise cancellation headphone. The feedback active noise cancellation headphone comprises a plurality of microphone sensors, an active noise controlling circuit, and speakers. A plurality of microphone sensors is used to detect environment noise in front of the speaker, and converts the environment noise to a noise perceiving signal and transmits the noise perceiving signal to the active noise controlling circuit. The active noise controlling circuit generates a noise cancellation signal according to the received noise perceiving signal, so that the speaker can generate a soundwave signal with a phase reversed to the environment noise for countering the low frequency environment noise.

In the preferred embodiment of the present invention, two or three microphone sensors may be evenly disposed on the peripheral area in front of the speaker. The noise perceiving signal sent to the active noise controlling circuit is generated by the microphone sensors, which are connected in parallel.

The present invention further provides a feedback active noise controlling circuit. The feedback active noise controlling circuit comprises a bandpass controller, an audio compensating circuit, an adder and a current converting repeater.

The bandpass controller receives the noise perceiving signal, which is obtained from the environment noise detected by the microphone sensors, and tunes the gain and the phase of the noise perceiving signal spectrum, so as to generate an environment noise signal.

The audio compensating circuit receives the audio input signal generated by the music apparatus, and generates an audio compensating signal whose high frequency attenuation is higher than its low frequency attenuation, so that it is capable of compensating the low frequency music to substantially reduce or eliminate the low frequency noise.

The adder comprises a first input terminal and a second input terminal whose gain can be separately adjusted. Wherein, the first input terminal is electrically coupled to the bandpass controller for receiving the environment noise signal mentioned above and for properly processing the received environment noise signal to generate a noise cancellation signal, which is used to drive the speaker for generating a soundwave signal with a phase reversed to the environment noise, so as to counter or reduce the low frequency environment noise. The second input terminal is electrically coupled to the audio compensating circuit for receiving the audio compensating signal mentioned above, and for amplifying the received audio compensating signal so as to generate an audio output signal, which is then transmitted to the speaker to output the music.

The current converting repeater receives a signal synthesized from the noise cancellation signal and the audio output signal, and converts it to a current signal for driving the speaker.

In the preferred embodiment of the present invention, the feedback active noise controlling circuit further comprises a power delay circuit. The power delay circuit receives a power supplied to the feedback active noise controlling circuit. The power delay circuit delays the power supply over a predetermined time before supplying the power to the current converting repeater when the power is turned on, so as to eliminate the weird sound that occurs when the power of the feedback active noise controlling circuit is turned on.

The power delay circuit mentioned above comprises a delay circuit and a transistor. The delay circuit is, for example, composed of a resistor and a capacitor, which are serially connected, for generating a delay control signal when the power is turned on. The transistor comprises a collector, an emitter, and a base, wherein the base is electrically coupled to the delay circuit mentioned above for receiving the delay control signal, and for delaying the power supply from the collector to the emitter in response to the delay control signal.

The feedback active noise controlling circuit further comprises a switch unit for controlling the power supplied to the feedback active noise controlling circuit. In the event when the power of the feedback active noise controlling circuit is cut off, the switch unit directs the audio input signal generated by the music apparatus to the speaker directly. Therefore, a user will be able to hear the music using the headphone even when the power of the feedback active noise controlling circuit has been cut off.

According to an aspect of the present invention, the audio compensating circuit of the feedback active noise controlling circuit comprises a first resistor, a second resistor, a first capacitor, a second capacitor, and a third resistor, wherein all of the components mentioned above comprise a first terminal and a second terminal, respectively. The first terminal of the first resistor receives the audio input signal generated by the music apparatus and outputs an audio compensating signal through the second terminal of the first capacitor. The components are connected in a way as follows. The second terminal of the first resistor is grounded, the first terminal of the second resistor is electrically coupled to the first terminal of the first resistor, the first terminal of the first capacitor is electrically coupled to the second terminal of the second resistor, the first terminal of the second capacitor is electrically coupled to the second terminal of the first capacitor, the first terminal of the third resistor is electrically coupled to the second terminal of the second capacitor and the second terminal of the third resistor is grounded.

According to another aspect of the present invention, the noise perceiving signal that is derived from environment noise is detected by a plurality of microphone sensors that are connected in parallel. In the feedback active noise controlling circuit of the present invention, the noise cancellation signal generated according to the noise perceiving signal is output to the speaker, so as to generate a soundwave signal with a phase reversed to the environment noise for countering or reducing the low frequency environment noise.

### BRIEF DESCRIPTION OF DRAWINGS

The accompanying drawings are included to provide a further understanding of the invention, and are incorporated in and constitute a part of this specification. The drawings illustrate embodiments of the invention, and together with the description, serve to explain the principles of the invention.

FIG. 1 is a schematic diagram illustrating the near-field effect of a feedback active noise cancellation headphone speaker.

FIG. 2 is a schematic diagram illustrating the measurement of the near-filed effect of the speaker disposed in a free (sound) field.

FIG. 3A and FIG. 3B are the schematic diagrams illustrating the layout scheme of two or three microphone sensors disposed in front of the speaker according to a preferred embodiment according to the present invention.

FIG. 4 is a schematic diagram illustrating the structure of two microphone sensors disposed in front of the speaker according to a preferred embodiment according to the present invention.

FIG. 5 is a schematic block diagram of a feedback active noise controlling circuit according to a preferred embodiment according to the present invention.

FIG. 6 is a schematic circuit diagram of an audio compensating circuit of the feedback active noise controlling circuit according to a preferred embodiment according to the present invention.

FIG. 7 is a schematic circuit diagram of a power supply and a switch unit of the feedback active noise controlling circuit according to a preferred embodiment according to the present invention.

### DETAILED DESCRIPTION

FIG. 1 is a schematic diagram illustrating the near-field effect of a feedback active noise cancellation headphone speaker. As shown in FIG. 1, when the microphone sensor 120 disposed in front of the speaker 110 collects a white noise from outside environment, a low frequency (about 50 Hz˜1 KHz) environment noise will be collected by the microphone sensor 120 due to the bandpass property in the earpiece chamber of headphone 140. Since the low frequency environment noise is characterized by its low frequency and long wavelength, the layout position of the microphone sensor 120 is not so critical.

However, after the microphone sensor 120 converts the collected low frequency environment noise into a noise perceiving signal, and transmits it to the active noise controlling circuit 130, the active noise controlling circuit 130 generates a noise cancellation signal according to the received noise perceiving signal and transmits it to the speaker 110, so as to generate a soundwave signal with a phase reversed to the environment noise for countering or reducing the low frequency environment noise detected by the microphone sensor 120. Since a sound energy vortex (flow) 150 is generated in front of the speaker 110 due to the near-field effect of such placement, and the microphone sensor 120 is disposed near to the neighborhood area in front of the speaker 110 and is just located inside the sound energy vortex (flow) 150 in front of the speaker 110. Therefore, due to the impact of the near-field effect, the microphone sensor 120 cannot clearly and accurately accept the low frequency environment noise in real time and is unable to transmit it to the active noise controlling circuit 130 to generate the inverse phase soundwave accurately for countering the low frequency noise.

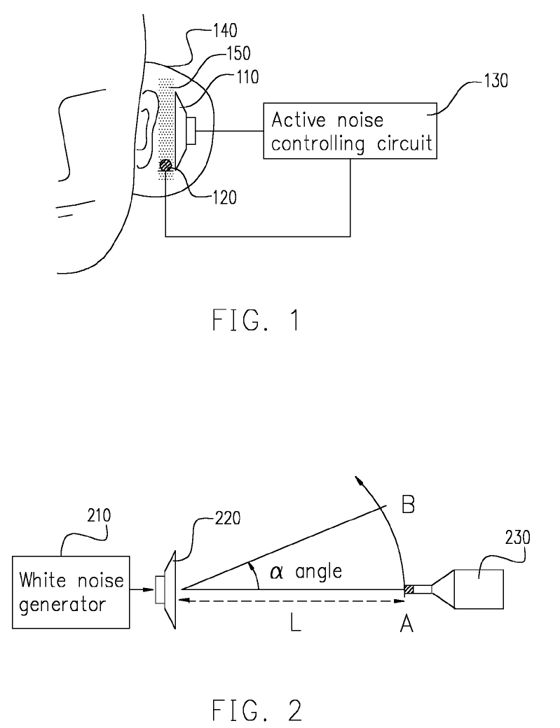

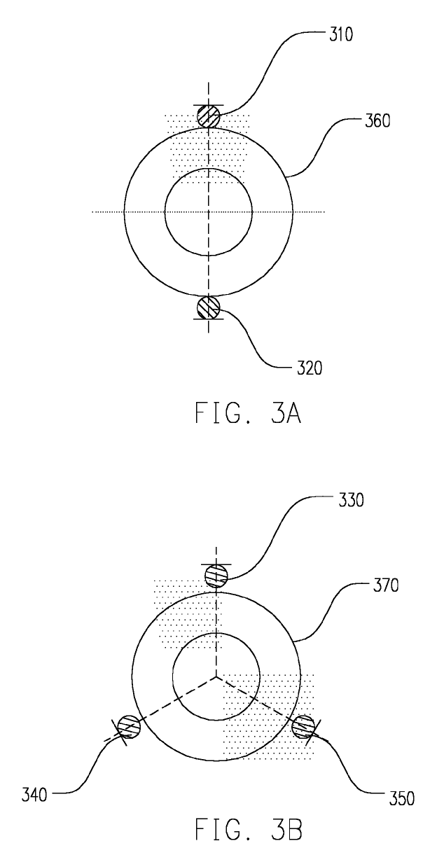

FIG. 2 is a schematic diagram illustrating the measurement of the near-filed effect of the speaker disposed in a free field. As shown in the diagram, a white noise generator 210 is used to simulate and generate a white noise signal, and the white noise signal is subsequently sent to the speaker 220 so as to generate a stable and evenly distributed white noise. Then, a sound level meter 230 measures the sound level (pressure) at different distance and angle α from the speaker 220, for example, measuring from the point A and point B as shown in the diagram, wherein the distance L of point A and point B from the speaker 220 is substantially same and inclined with each other by an angle α. From the experiment mentioned above, the equal stable sound levels (pressures) are measured from different angles in front of the speaker 220 at a distance, for example, greater than the diameter of the speaker 220. However, the different unstable sound levels (pressures) are measured from different angles in front of the speaker 220 apart from the speaker at a distance less than the diameter of the speaker 220, for example, within range of 5˜10 mm from the speaker 220. The above experiment provides the evidence of the existence and the impact of the near-field effect occurring in the neighborhood area in front of the speaker 220.

FIG. 3A and FIG. 3B are the schematic diagrams illustrating the layout scheme of two or three microphone sensors disposed in front of the speaker according to a preferred embodiment of the present invention. As shown in the figures, in order to resolve the problem mentioned above, the present invention disposes two microphone sensors 310 and 320 in front of the speaker 360 as shown in FIG. 3A, or disposes three microphone sensors 330, 340, and 350 in front of the speaker 370 as shown in FIG. 3B. The sound reception direction of the microphone sensors 310, 320, 330, 340, and 350 aims to the center line in front of the speaker. Since the microphones 310, 320, 330, 340 and 350 are positioned in different locations, the plurality of microphone sensors 310 and 320 or 330, 340, and 350 respectively receive the signals having different levels of clearness, so that they can be compensated with each other to improve the sound reception quality of the microphone sensors 310 and 320 or 330, 340, and 350. Accordingly, the active noise controlling circuit is able to generate an inverse phase soundwave more accurately for countering the low frequency noise, and further to improve the noise reduction performance of the active noise cancellation headphone.

As the embodiments shown in FIG. 3A and FIG. 3B, the two microphone sensors 310 and 320 are positioned symmetrically in front of the speaker 360 as shown in FIG. 3A, or three microphone sensors 330, 340, and 350 are disposed evenly in front of the speaker 370 as shown in FIG. 3B. However, it will be apparent to one of the ordinary skill in the art that this only serves a preferable placement herein and should not be restricted to as the only placement of the microphone sensors. In fact, the placement can be of different angles and distances as desired by the designer based on different requirements.

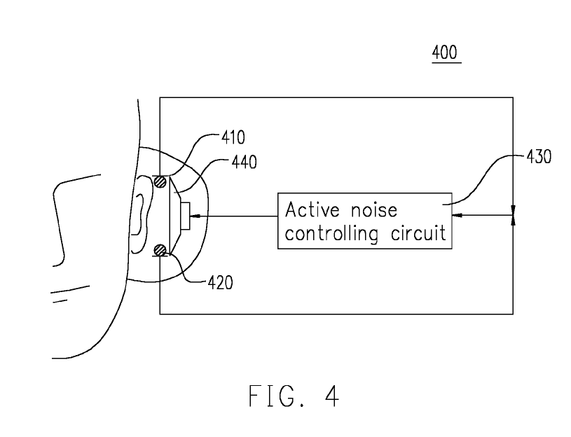

FIG. 4 is a schematic diagram illustrating the structure of two microphone sensors disposed in front of the speaker according to the preferred embodiment of the present invention. As shown in FIG. 4, the feedback active noise cancellation headphone 400 comprises two microphone sensors 410 and 420, an active noise controlling circuit 430, and a speaker 440. Two microphone sensors 410 and 420 disposed in the peripheral area in front of the speaker 440 detect the environment noise, and convert the environment noise into a noise perceiving signal, which is then sent to the active noise controlling circuit 430. The active noise controlling circuit 430 generates a noise cancellation signal according to the received noise perceiving signal so that the speaker 440 can generate a soundwave signal with a phase reversed to the environment noise for countering or reducing the low frequency environment noise. The advantage of this method has been described hereinbefore, in that two microphone sensors 410 and 420 that are disposed at different locations accept the signals having different levels of clearness for compensating with each other so as to improve the overall sound reception quality of the microphone sensors 410 and 420 as a whole. Accordingly, the active noise controlling circuit 430 is able to generate more accurate and more effective inverse phase soundwave for countering the low frequency noise, and further to improve the noise reduction performance of the active noise cancellation headphone.

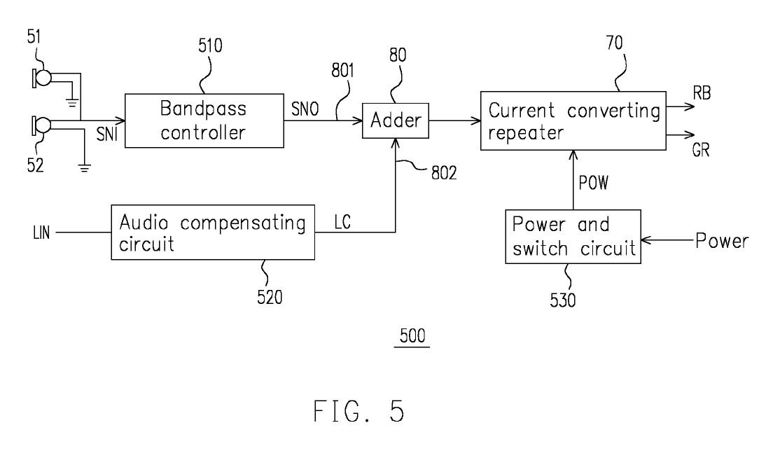

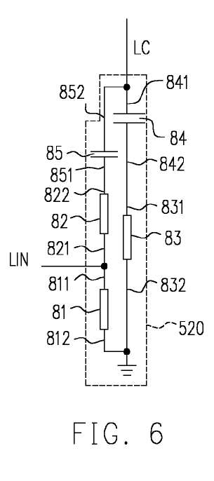

FIG. 5 is a schematic block diagram of a feedback active noise controlling circuit of the preferred embodiment according to the present invention. As shown in the diagram, the feedback active noise controlling circuit 500 comprises a bandpass controller 510, an audio compensating circuit 520, an adder 80, a current converting repeater 70, and a power and switch circuit 530.

The bandpass controller 510 receives a noise perceiving signal SNI, which is generated by a plurality of microphone sensors 51 and 52 that are connected in parallel upon detecting environmental noise. Next, the bandpass controller 510 tunes the gain and the phase of the noise perceiving signal SNI to generate an environment noise signal SNO which is then output to the first input terminal 801 of the adder 80 where the environment noise signal SNO is amplified into a noise cancellation signal. Then, the noise cancellation signal is further converted into a current signal by the current converting repeater 70 to drive the speaker through the RB/GR transmission line, so that the speaker can generate a soundwave signal with a phase reversed to the environment noise for countering or reducing the low frequency environment noise.

The second input terminal 802 of the adder 80 receives an audio input signal LIN generated by the music apparatus (not shown), so that the expected music can be output from the speaker. Further, the gain of the second input terminal 802 and the first input terminal 801 mentioned above of the adder 80 can be separately adjusted, so that the sound level of the music is not impacted when tuning the gain of the environment noise signal SNO to improve the noise reduction efficiency.

However, since the noise perceiving signal SNI derived from the environment noise detected by the plurality of microphone sensors 51 and 52 usually comprises the music expected to be heard by the user, the 100 Hz˜1K Hz musicmay also be partially eliminated in accompanying with the noise signal. In order to prevent this phenomenon and the user can hear the music with the original quality and does not recognize any change of the music while the operation of the feedback active noise controlling circuit 500 is performed, an audio compensating circuit 520 is added prior to the second input terminal 802 of the adder 80 where the audio input signal LIN is input, so that the music which may be partially eliminated can be compensated. The compensating method is described as follows. The audio input signal LIN generated by the music apparatus is received first, and an audio compensating signal LC whose high frequency attenuation is higher than its low frequency attenuation is generated by the audio compensating circuit 520, so as to compensate the attenuation of the low frequency music, and it is then input into the second input terminal 802 of the adder 80.

As shown in FIG. 6, the audio compensating circuit 520 comprises a first resistor 81, a second resistor 82, a first capacitor 85, a second capacitor 84 and a third resistor 83, wherein all of the components mentioned above comprise a first terminal 811, 821, 851, 841, 831 and a second terminal 812, 822, 852, 842, 832, respectively. The first terminal 811 of the first resistor 81 receives the audio input signal LIN generated by the music apparatus, and outputs an audio compensating signal LC through the second terminal 852 of the first capacitor 85. The above components are connected in a way as follows. The second terminal 812 of the first resistor 81 is grounded, the first terminal 821 of the second resistor 82 is electrically coupled to the first terminal 811 of the first resistor 81, the first terminal 851 of the first capacitor 85 is electrically coupled to the second terminal 822 of the second resistor 82, the first terminal 841 of the second capacitor 84 is electrically coupled to the second terminal 852 of the first capacitor 85, the first terminal 831 of the third resistor 83 is electrically coupled to the second terminal 842 of the second capacitor 84 and the second terminal 832 of the third resistor 83 is grounded.

Therefore, the feedback active noise controlling circuit 500 can compensate the music that may be partially eliminated. Further, since the gain of the audio input signal LIN and the noise perceiving signal SNI can be separately tuned, the gain of the audio input signal LIN is not impacted by the adjustment of the anti-noise gain. Therefore, it can provide a stable sound level music regardless of the level of the sound volume. In other words, the low frequency cracked sound will not occur even when the level of the sound is increased.

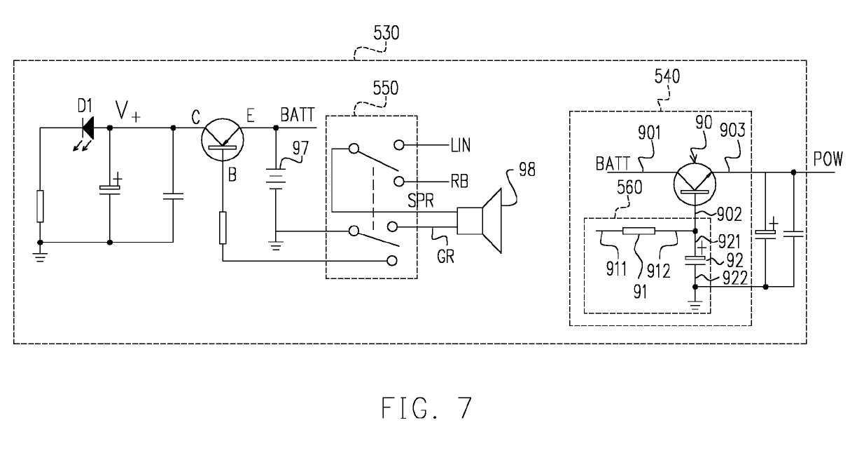

Further, the present invention provides a solution to resolve the problem that the weird tone is output from the speaker as the circuit is not in a steady state at the moment when the power is just being turned on. As shown in FIG. 5, the feedback active noise controlling circuit 500 further comprises a power and switch circuit 530, wherein the power and switch circuit 530 comprises a power delay circuit 540 as shown in FIG. 7 for accepting a power BATT, which is supplied to the feedback active noise controlling circuit 500, and delaying a predetermined period of time before supplying a power POW to the current converting repeater 70 when the power V+ is turned on. The detailed operation principle is described hereinafter.

As shown in FIG. 7, the power delay circuit 540 comprises a transistor 90, and a delay circuit 560 composed of a resistor 91 and a capacitor 92 that are serially connected. The transistor 90 comprises a collector 901, an emitter 903, and a base 902, and the resistor 91 comprises a first terminal 911 and a second terminal 912, the capacitor 92 comprises a first terminal 921 and a second terminal 922. Wherein, the collector 901 is electrically coupled to a power BATT supplied by a battery 97, the base 902 is electrically coupled to the second terminal 912 of the resistor 91 and the first terminal 921 of the capacitor 92, and the second terminal 922 of the capacitor 92 is grounded.

When the power V+ electrically coupled to the first terminal 911 of the resistor 91 is turned on (i.e. V+ supplies the required power to other circuits), the capacitor 92 is charged so as to generate a delay control signal. The delay control signal delays the power supply over a predetermined period of time before turning on the transistor 90, so that the power POW output from the emitter 903 of the transistor 90 is delayed supplying to the current converting repeater 70 shown in FIG. 5. Therefore, when the power V+ is turned on, the feedback active noise controlling circuit 500 does not output the instant weird tone from its speaker anymore.

As shown in FIG. 7, the power and switch circuit 530 further comprises a switch unit 550. The switch unit 550 is used to control whether or not to turn on the power V+ which is supplied to the feedback active noise controlling circuit 500, and to have the audio input signal LIN generated by the music apparatus (not shown) directly pass to the speaker 98 when the power V+ to the feed back active noise controlling circuit is cut off by the switch unit 550, so that the headphone can be used to hear the music when the power V+ of the feedback active noise controlling circuit 500 has been cut off.

An experiment is conducted to confirm the effect of the noise reduction of the feedback active noise cancellation headphone which uses a plurality of microphone sensors. The feedback active noise cancellation headphone 400 shown in FIG. 4 is put on an artificial head device, which is capable of measuring the received noise sound levels of various frequencies, and the measurement results are recorded in Table 1 and Table 2. Table 1 shows the measurement results in a case when only one microphone sensor 410 connected to the active noise controlling circuit 430, and Table 2 shows the measurement results in a case when both the microphone sensors 410 and 420 are simultaneously connected to the active noise controlling circuit 430. Wherein, the field ANC-OFF shows the measurement values when the operation of the active noise controlling circuit 430 is turned off, that is, the sound levels of the noises heard by the artificial head device when the noises are not eliminated, and the field ANC-ON in Table 1 shows the sound levels of the noises heard by the artificial head device when only one microphone sensor 410 is connected to the active noise controlling circuit 430. The figures shown in Table 2 are the sound levels of the noises heard by theartificial head device when both the microphone sensors 410 and 420 are simultaneously connected to the active noise controlling circuit 430. The last row of Table 1 and Table 2 show the average value of the noise reduction amount, respectively. Referring to the average value of the noise reduction amount, the average value of the noise reduction amount in the case when only one microphone sensor 410 is connected to the active noise controlling circuit 430 is 9.42467754 dB, and the average value of the noise reduction amount in the case when both the microphone sensors 410 and 420 are simultaneously connected to the active noise controlling circuit 430 is 12.7675294 dB. Therefore, by using the two-microphone sensors design and cooperating with the feedback active noise controlling circuit 500 of the present invention, the noise reduction effect is significantly improved.

TABLE 1

| Frequency | ANC-OFF | ANC-ON | Noise reduction |
|---|---|---|---|
| (Hz) | noise amount (dB) | noise amount (dB) | amount |
| 50 | −44.892052 | −46.355553 | 1.463501 |
| 63 | −47.250725 | −51.611275 | 4.36055 |
| 80 | −46.059258 | −52.916901 | 6.857643 |
| 100 | −39.596458 | −50.056454 | 10.46 |
| 125 | −40.698879 | −52.588493 | 11.88961 |
| 160 | −44.13002 | −57.5037 | 13.37368 |
| 200 | −49.081154 | −58.509605 | 9.428451 |
| 250 | −51.771255 | −60.032673 | 8.261418 |
| 315 | −59.943424 | −69.942879 | 9.999455 |
| 400 | −68.614731 | −79.727463 | 11.11273 |
| 500 | −72.215195 | −83.750633 | 11.53544 |
| 630 | −73.721779 | −82.608246 | 8.886467 |
| 800 | −72.781471 | −79.317261 | 6.53579 |
| 1000 | −79.337273 | −76.014885 | −3.32239 |
| Average | | | 9.42467754 |

TABLE 2

| Frequency | ANC-OFF | ANC-ON | Noise reduction |
|---|---|---|---|
| (Hz) | noise amount (dB) | noise amount (dB) | amount |
| 50 | −59.613277 | −61.625042 | 2.011765 |
| 63 | −59.073704 | −64.525406 | 5.451702 |
| 80 | −54.281155 | −61.29026 | 7.009105 |
| 100 | −47.093666 | −57.906025 | 10.81236 |
| 125 | −42.541756 | −57.877411 | 15.33566 |
| 160 | −44.581146 | −62.431255 | 17.85011 |
| 200 | −42.310223 | −59.130478 | 16.82026 |
| 250 | −51.757565 | −63.474697 | 11.71713 |
| 315 | −57.003044 | −68.348465 | 11.34542 |
| 400 | −63.156078 | −75.24823 | 12.09215 |
| 500 | −63.727406 | −79.12429 | 15.39688 |
| 630 | −71.959145 | −83.755692 | 11.79655 |
| 800 | −69.567673 | −76.19136 | 6.623687 |
| 1000 | −77.687004 | −73.204292 | −4.48271 |
| Average | | | 12.7675294 |

Although the invention has been described with reference to a particular embodiment thereof, it will be apparent to one of the ordinary skill in the art that modifications to the described embodiment may be made without departing from the spirit of the invention. Accordingly, the scope of the invention will be defined by the attached claims not by the above detailed description.

1. A feedback active noise controlling circuit, comprising:
a bandpass controller for receiving a noise perceiving signal generated upon detection of an environment noise, for tuning a gain and a phase of a spectrum of the noise perceiving signal so as to generate an environment noise signal;
an audio compensating circuit for receiving an audio input signal so as to generate an audio compensating signal whose high frequency attenuation is higher than its low frequency attenuation;
an adder comprising a first input terminal and a second input terminal whose gain can be separately adjusted, wherein the first input terminal is electrically coupled to the bandpass controller for receiving the environment noise signal, and amplifying the environment noise signal to generate a noise cancellation signal, and the second input terminal is electrically coupled to the audio compensating circuit for receiving the audio compensating signal, and amplifying the audio compensating signal to generate an audio output signal; and
a current converting repeater electrically coupled to the adder for receiving a signal synthesized from the noise cancellation signal and the audio output signal, and converting the signal into a current signal for driving a speaker.

2. The feedback active noise controlling circuit of claim 1, further comprising a power delay circuit for receiving a power supplied to the feedback active noise controlling circuit, and delaying the power supply to the current converting repeater for a predetermined period of time when the power is turned on.

3. The feedback active noise controlling circuit of claim 2, wherein the power delay circuit comprises:
a delay circuit that is configured to generate a delay control signal when the power is turned on; and
a transistor comprising a collector, an emitter, and a base, wherein the base is electrically coupled to the delay circuit for receiving the delay control signal, and delaying a power supply over a predetermined period of time before turning on the power received by the collector to the emitter according to the delay control signal.

4. The feedback active noise controlling circuit of claim 3, wherein the delay circuit comprises a resistor and a capacitor that are serially connected.

5. The feedback active noise controlling circuit of claim 1, further comprising a switch unit that is configured to control a power supplied to the feedback active noise controlling circuit, and have the audio input signal directly pass to the speaker and then output the audio input signal from the speaker when the power is cut off.

6. The feedback active noise controlling circuit of claim 1, wherein the audio compensating circuit comprises:
a first resistor comprising a first terminal and a second terminal, wherein the first terminal of the first resistor receives the audio input signal, and the second terminal of the first resistor is grounded;
a second resistor comprising a first terminal and a second terminal, wherein the first terminal of the second resistor is electrically coupled to the first terminal of the first resistor;
a first capacitor comprising a first terminal and a second terminal, wherein the first terminal of the first capacitor is electrically coupled to the second terminal of the second resistor, and the second terminal of the first capacitor outputs the audio compensating signal;
a second capacitor comprising a first terminal and a second terminal, wherein the first terminal of the second capacitor is electrically coupled to the second terminal of the first capacitor; and
a third resistor comprising a first terminal and a second terminal, wherein the first terminal of the third resistor is electrically coupled to the second terminal of the second capacitor, and the second terminal of the third resistor is grounded.

7. The feedback active noise controlling circuit of claim 1, wherein the noise perceiving signal is generated by sensing the environment noise using a plurality of microphone sensors that are connected in parallel.

8. A feedback active noise cancellation headphone, comprising:
a speaker for receiving a noise cancellation signal and generate a soundwave signal with a phase reversed to an environment noise;
a plurality of microphone sensors for detecting the environment noise positioned in front of the speaker, and for converting the environment noise into a noise perceiving signal; and
an active noise controlling circuit electrically coupled to the microphone sensors and the speaker for receiving the noise perceiving signal, for generating the noise cancellation signal according to the noise perceiving signal.

9. The feedback active noise cancellation headphone of claim 8, wherein the plurality microphone sensors is comprised of two microphone sensors.

10. The feedback active noise cancellation headphone of claim 9, wherein the two microphone sensors are disposed symmetrically in front of the speaker.

11. The feedback active noise cancellation headphone of claim 8, wherein the plurality of microphone sensors is comprised of three microphone sensors.

12. The feedback active noise cancellation headphone of claim 11, wherein the three microphone sensors are disposed evenly in front of the speaker.

13. The feedback active noise cancellation headphone of claim 8, wherein the noise perceiving signal is generated by sensing the environment noise using the microphone sensors that are connected in parallel.

14. The feedback active noise cancellation headphone of claim 8, wherein the microphone sensors are disposed in front of the speaker.
